(19) United States
(12) Patent Application Publication
(10) Pub. No.: US 2020/0170624 A1
(43) Pub. Date: Jun. 4, 2020

(54) DIAGNOSTIC APPARATUS AND DIAGNOSTIC METHOD

(71) Applicant: Hitachi, Ltd., Tokyo (JP)

(72) Inventors: Yoshimi NOGUCHI, Tokyo (JP); Maki KUWAYAMA, Tokyo (JP); Yoshiko YAMAMOTO, Tokyo (JP); Noriko ITABASHI, Tokyo (JP); Naoyuki MURAYAMA, Tokyo (JP); Yoko FUJIHARA, Tokyo (JP)

(21) Appl. No.: 16/695,503

(22) Filed: Nov. 26, 2019

(30) Foreign Application Priority Data
Nov. 30, 2018 (JP) 2018-225650

(51) Int. Cl. A61B 8/08; A61B 8/14; A61B 8/00; G06T 7/00; G06K 9/62

(52) U.S. Cl. CPC A61B 8/5223; A61B 8/14; A61B 8/463; A61B 8/0825; G06T 2207/10132; G06K 9/623; G06T 2207/30096; G06K 2209/05; G06T 7/0012

(57) ABSTRACT

A diagnostic apparatus diagnosing by using a tomographic image of a subject, comprising: an image generator configured to generate the tomographic image based on data obtained from the subject; and a detector configured to perform a process to detect a lesion from the tomographic image, the detector being configured to: generate, using the tomographic image, a filter map for extracting a tissue lesion that is potentially abnormal from the tomographic image; and detect a lesion included in the tomographic image, using the tomographic image and the filter map, and output detection information including a detection result of the lesion.

### CLAIM OF PRIORITY

The present application claims priority from Japanese patent application JP 2018-225650 filed on Nov. 30, 2018, the content of which is hereby incorporated by reference into this application.

### BACKGROUND OF THE INVENTION

The present invention relates to an image diagnostic apparatus and an image diagnostic method in the medical field.

In breast cancer screening, a mammogram is generally performed. However, the mammogram has a problem of having a lower detection rate of a lesion in examinees for the dense breast, which occurs more often in Asian women. To solve this problem, ultrasound is added to the mammogram to improve the detection rate of a legion.

In ultrasound, a large number of ultrasound images need to be read, and also this technology has problems such as putting a large burden on an examiner and variations in the detection rate of a lesion depending on an examiner. Thus, the advancement of CAD (computer-aided diagnosis/detection) technology is expected.

The technology described in Japanese Patent Application Laid-open Publication No. 2015-154918 is known as one of the CAD technologies. Japanese Patent Application Laid-open Publication No. 2015-154918 describes an apparatus including a possible lesion detection stage for detecting possible lesions in a medical image, a peripheral object detection stage for detecting an anatomical object in the medical image, a possible lesion examination stage for examining the possible lesions based on anatomical context information including information regarding a relationship between the position of the possible lesions and the position of the anatomical object, and a false positive removal stage for removing false positive lesions from possible lesions based on the examination results.

### SUMMARY OF THE INVENTION

There is a need to automatically obtain the lesion detection result during the screening, using an ultrasound diagnosis apparatus that has an excellent real-time characteristic.

With the technology described in Japanese Patent Application Laid-open Publication No. 2015-154918, it is necessary to generate a detector by executing machine leaning in advance. In order to improve the accuracy of the detector, a large amount of learning data is required, and also, it is necessary to configure the most appropriate algorithm. Furthermore, the processing time is long and the process cost is high in detecting a lesion or the like. As a result, the technology described in Japanese Patent Application Laid-open Publication No. 2015-154918 might not be able to fully address the above-mentioned need.

The present invention is aiming at providing an apparatus and a method that can detect a lesion automatically with a high degree of accuracy in diagnosis using ultrasound images.

A representative example of the present invention disclosed in this specification is as follows: a diagnostic apparatus diagnosing by using a tomographic image of a subject, comprises an image generator configured to generate the tomographic image based on data obtained from the subject; and a detector configured to perform a process to detect a lesion from the tomographic image. The detector being configured to: generate, using the tomographic image, a filter map for extracting a tissue lesion that is potentially abnormal from the tomographic image; and detect a lesion included in the tomographic image, using the tomographic image and the filter map, and output detection information including a detection result of the lesion.

According to one embodiment of the present invention, a diagnosis apparatus can detect a lesion automatically with a high degree of accuracy. Other problems, configurations, and effects than those described above will become apparent in the descriptions of embodiments below.

### BRIEF DESCRIPTION OF THE DRAWINGS

The present invention can be appreciated by the description which follows in conjunction with the following figures, wherein:

FIG. 1 is a diagram illustrating a configuration example of an ultrasound diagnostic apparatus of Embodiment 1;

FIG. 2 is a diagram illustrating an example of a tomographic image generated by the ultrasound diagnostic apparatus of Embodiment 1;

FIG. 3 is a flowchart for explaining the process performed by a lesion detector of Embodiment 1;

FIGS. 4A, 4B, and 4C are diagrams for explaining an example of the method to generate a filter map of Embodiment 1;

FIG. 5 is a diagram illustrating an example of the flow of the process performed by the lesion detector of Embodiment 1.

FIG. 6 is a diagram illustrating examples of a lesion detection result presented by a display unit of Embodiment 1;

FIG. 7 is a diagram illustrating a configuration example of an ultrasound diagnostic apparatus of Embodiment 2;

FIGS. 8A and 8B are diagrams illustrating how the ultrasound diagnostic apparatus of Embodiment 2 analyzes the shape of a lesion;

FIG. 9 is a diagram illustrating how the ultrasound diagnostic apparatus of Embodiment 2 determines whether a lesion is benign or malignant, as well as the category thereof; and

FIGS. 10A, 10B, and 10C are diagrams illustrating an example of a screen displayed by the display unit of Embodiment 3.

### DETAILED DESCRIPTION OF THE EMBODIMENTS

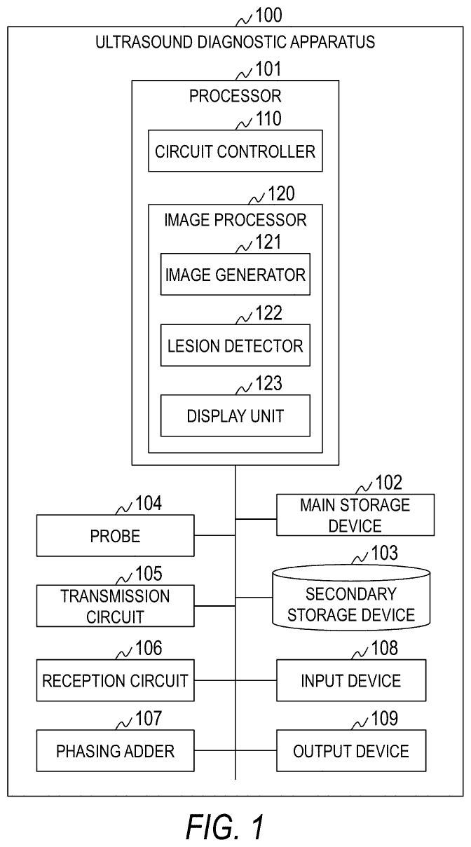

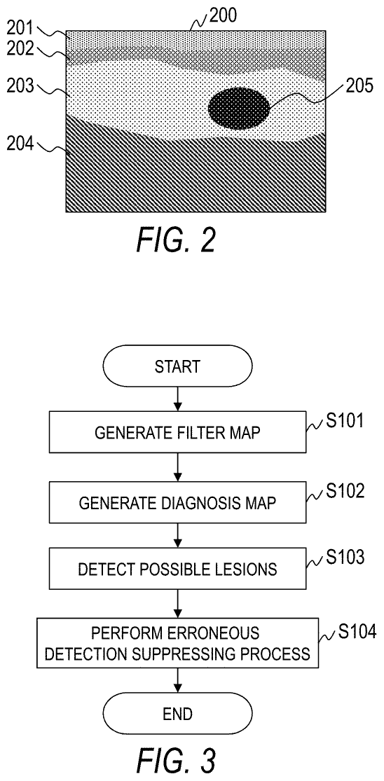

FIG. 1 is a diagram illustrating a configuration example of an ultrasound diagnostic apparatus of Embodiment 1. FIG. 2 is a diagram illustrating an example of a tomographic image generated by the ultrasound diagnostic apparatus of Embodiment 1.

The ultrasound diagnostic apparatus 100 outputs ultrasound to a subject, and generates a tomographic image (echo image) from the reflected ultrasound signal (echo signal). The ultrasound diagnostic apparatus 100 also performs a detection process to detect a lesion from the tomographic image, and presents the detection result of a lesion to the user such as a medical staff.

The ultrasound diagnostic apparatus 100 includes a CPU 101, a main storage device 102, a secondary storage device 103, a probe 104, a transmission circuit 105, a reception circuit 106, a phasing adder 107, an input device 108, and an output device 109. The respective pieces of hardware are connected to each other via a bus or the like.

The CPU 101 executes programs stored in the main storage device 102. The CPU 101 operates as function units (modules) that realize specific functions, respectively, by executing processes in accordance with the programs. In the descriptions below, when the process is described using a module as the subject, that means that the CPU 101 is executing the program that realizes such a module. The CPU 101 of this embodiment operates as the circuit controller 110 and the image processor 120. Each module will be explained in detail later.

The main storage device 102 is a storage device such as a memory, and stores therein programs to be executed by the CPU 101 and information. The secondary storage device 103 is a storage device such as a hard disk drive (HDD) and solid-state drive (SSD), and stores data permanently.

In the main storage device 102 of this embodiment, the programs that realize the circuit controller 110 and the image processor 120 are stored. In the secondary storage device 103 of this embodiment, tomographic images, subject information, and the like are stored. The subject information includes the age, gender, or the like of subjects.

The programs and information stored in the main storage device 102 may also be stored in the secondary storage device 103. In this case, the CPU 101 reads out the program or information from the secondary storage device 103, loads it onto the main storage device 102, and executes the program loaded onto the main storage device 102.

The storage device for storing data and information can be changed as appropriate according to the purpose of use, processing performance, storage capacity, and the like.

The probe 104 generates ultrasound, receives ultrasound that bounces back from the inside of the subject, and converts the received ultrasound into an echo signal. The probe 104 has an ultrasound transducer that generates ultrasound. There are no limitations on the form of the probe 104 as long as it can receive the ultrasound. Possible examples of the probe include a general hand-held probe, or an automatic scanning probe such as ABUS (automatic breast ultrasound system).

The transmission circuit 105 outputs a transmission signal of ultrasound to the probe 104 at a certain interval. The reception circuit 106 receives an echo signal from the probe 104.

The phasing adder 107 performs phasing addition on a time-series echo signal, thereby generating time-series RF signal frame data. The phasing adder 107 is equipped with an analog-digital (AD) converter. The RF signal frame data is stored in the main storage device 102 or the secondary storage device 103 as observation data.

The input device 108 is used by the user to input information, and examples thereof include a keyboard, mouse, touch panel, and button.

The output device 109 is used to output information to the user, and examples thereof include a display, printer, and speaker. The output device 109 of this embodiment outputs tomographic images, lesion detection results, and the like.

Below, the circuit controller 110 and the image processor 120 will be explained.

The circuit controller 110 is configured to control the transmission circuit 105 and the reception circuit 106. For example, the circuit controller 110 controls the transmission circuit 105 to adjust the radiation direction of ultrasound, the output interval of the transmission signal, and the like.

The image processor 120 generates tomographic images using the RF signal frame data generated by the phasing adder 107, and performs an image process on the tomographic images such as a filtering process and lesion detection process. The image processor 120 also stores the lesion detection result and the like into the secondary storage device 103, together with the tomographic images. The lesion detection result includes presence or absence of a lesion, the position of the lesion in an image, a detection time, and the like. In a case where the probe 104 is equipped with a sensor for detecting positions such as a magnetic sensor, the lesion detection result may include spatial position information.

The image processor 120 generates a tomographic image 200 as illustrated in FIG. 2, for example. In the tomographic image 200, a layer 201 represents the layer corresponding to skin, a layer 202 represents the layer corresponding to fat, a layer 203 represents the layer corresponding to mammary gland, and a layer 204 represents the layer corresponding to the pectoralis major. An object 205 represents a lesion.

The image processor 120 of this embodiment is constituted of an image generator 121, a lesion detector 122, and a display unit 123. The image processor 120 may also have other modules.

The image generator 121 generates a tomographic image by performing a scanning conversion process on the RF signal frame data. The image generator 121 stores the generated tomographic image in the secondary storage device 103. The scanning conversion process is a known technology and the detailed description thereof is therefore omitted.

The lesion detector 122 performs a detection process to detect a lesion from the tomographic image, and outputs the detection result as detection information. The lesion detector 122 stores, in the secondary storage device 103, the detection information corresponding to the tomographic image. The process performed by the lesion detector 122 will be described in detail later.

The display unit 123 generates display data to present the tomographic image, the lesion detection result, and the like. The method to present the lesion detection result will be explained in detail later.

The circuit controller 110, the probe 104, the transmission circuit 105, the reception circuit 106, and the phasing adder 107 function as an observation module to observe the subject using ultrasound, and the image processor 120, the main storage device 102, the secondary storage device 103, the input device 108, and the output device 109 function as a data processing module to process images. The observation unit and the data processing unit may be realized using different devices.

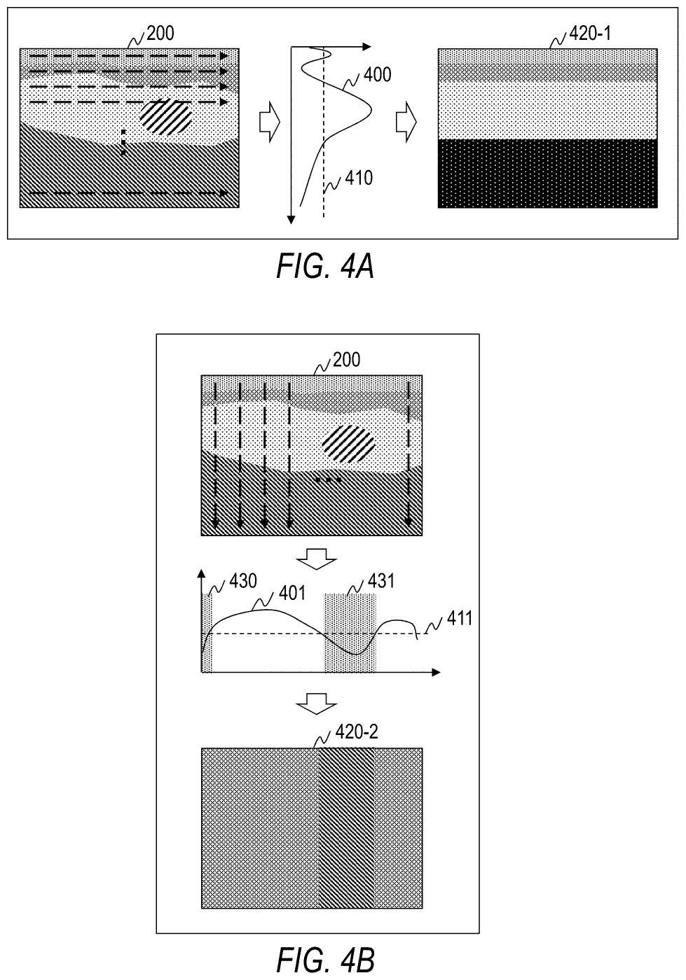

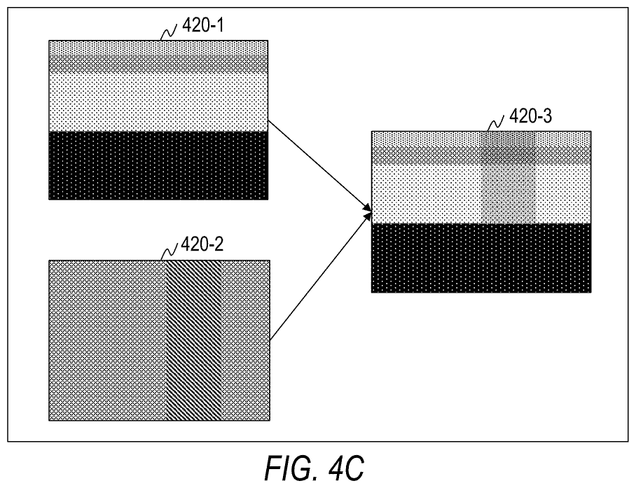

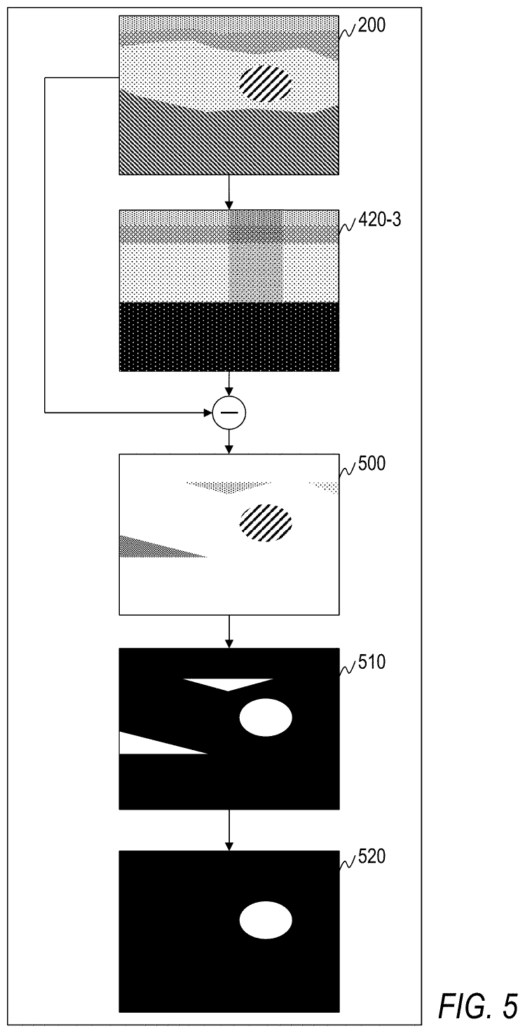

FIG. 3 is a flowchart for explaining the process performed by the lesion detector 122 of Embodiment 1. FIGS. 4A, 4B, and 4C are diagrams for explaining an example of the method to generate a filter map of Embodiment 1. FIG. 5 is a diagram illustrating an example of the flow of the process performed by the lesion detector 122 of Embodiment 1.

In a case where a tomographic image is generated by the image generator 121, the lesion detector 122 starts the process described below. The timing to start the process is not limited to that of this embodiment. The process may be performed after one tomographic image is generated, or the process may be performed after a certain number of tomographic images are accumulated.

The lesion detector 122 generates one filter map using one tomographic image (Step S101). Specifically, the processes described below are performed.

(Process A1) The lesion detector 122 sets analysis layers of appropriate depths at an appropriate angle and an appropriate interval with respect to a tomographic image. The angle of the analysis layer is determined based on the orientation of the tomographic image or the orientation of the beam. The depth and interval of the analysis layers is determined based on the detection accuracy, the processing cost, and the like. Below, the angle, depth and interval are collectively described as an analysis condition.

FIG. 4A shows a state in which analysis layers each having a depth of one pixel are set in parallel with the horizontal direction of the tomographic image at one-pixel intervals. That is, the number of the analysis layers is the same as the number of pixels in the vertical direction. FIG. 4B shows a state in which analysis layers having a depth of one pixel are set in parallel with the vertical direction of the tomographic image at one-pixel intervals. That is, the number of the analysis layers is the same as the number of pixels in the horizontal direction.

(Process A2) The lesion detector 122 calculates an average value of feature amounts of a plurality of pixels included in the analysis layer for each analysis layer. The lesion detector 122 calculates the distribution of the average values of the feature amounts in the respective analysis layers. Furthermore, in order to remove the high-frequency component, the lesion detector 122 performed a smoothing filter process on the distribution of the average values of the feature amounts to calculate a smoothed distribution.

Examples of the feature amount of the tomographic image include brightness, dispersion, texture, and co-occurrence feature. It is also possible to calculate an average value of combination values of a plurality type of feature amounts. In this embodiment, brightness is used for the feature amount. Examples of the smoothing filter include a moving average filter, Gaussian filter, and median filter. It is also possible to combine a plurality of smoothing filters.

The lesion detector 122 may also calculate the average value of the feature amounts after converting the tomographic image to an image constituted of prescribed feature amounts. An example thereof is a process to convert the tomographic image to an image with inverted brightness.

A smoothed distribution 400 is calculated from the analysis layers shown in FIG. 4A, and a smoothed distribution 401 is calculated from the analysis layers shown in FIG. 4B. The horizontal axis is the analysis layer, and the vertical axis is the average value of the feature amounts of the analysis layer.

(Process A3) The lesion detector 122 generates a filter map by mapping the smoothed distribution onto an image having the same size as the tomographic image. Specifically, the lesion detector 122 sets the average value of the feature amounts in the analysis layer to the pixels included in one analysis layer. The corresponding relationship between an analysis layer and a pixel row is determined based on the orientation, depth, and number of the analysis layer. In FIGS. 4A and 4B, one analysis layer corresponds to one pixel row.

A filter map 420-1 is generated from a tomographic image set with the analysis layers of FIG. 4A, and a filter map 420-2 is generated from a tomographic image set with the analysis layers of FIG. 4B.

The lesion detector 122 may also have a threshold value 410 for the smoothed distribution 400 to narrow down the regions to be detected. Specifically, the lower section of the tomographic image 200 are the layers corresponding to the greater pectoral muscle and lung region, and the brightness is low. Thus, the lesion detector 122 excludes regions that do not reach the threshold value 410 (analysis layer group) from the regions to be detected. The lesion detector 122 may also set a threshold value 411 for the smoothed distribution 401, detect a shadow region 430 caused by a nipple or a lesion, and a shadow region 431 caused by the probe 104 not in contact with the breast, and excludes the regions 430 and 431 from the filter map 420-2. The threshold value may be set in advance, or the average value of the smoothed distribution 401 may be used for the threshold value.

The lesion detector 122 may also generate a combined filter map by combining filter maps generated from a plurality of analysis layers having different analysis conditions. In this case, the feature amounts to be addressed can be changed for each analysis condition. In a case where combining filter maps generated from the same feature amount, the lesion detector 122 performs weighted addition operation to generate a combined filter map. For example, by performing the weighted addition operation on the filter maps 420-1 and 420-2, a combined filter map 420-3 is generated. In the descriptions below, when it is not necessary to distinguish a combined filter map from a filter map, the combined filter map will simply be referred to as a filter map.

As illustrated in FIG. 2, the inside of the human body has a layered structure. The average value of the feature amounts of the group of pixels in the analysis layer is a value characterizing the layered structure. Because there are only a small number of pixels that corresponds to a lesion among the group of pixels included in the analysis layer, the average value of the feature amounts of the group of pixels included in the analysis layer is not affected by a lesion almost at all. Thus, the filter map can be handled as an image representing the normal tissue regions of the human body.

The descriptions above are for the process of Step S101.

Next, the lesion detector 122 generates a diagnosis map using the tomographic image and the filter map (Step S102). The diagnosis map is an image obtained by removing normal tissue regions from the tomographic image, in other words, an image showing a tissue region deemed abnormal.

Specifically, the lesion detector 122 generates a diagnosis map by calculating a difference between the feature amount in each pixel of the tomographic image and the feature amount of each pixel of the filter map. The filter map is an image showing normal tissue regions of human body. Thus, by calculating the difference between the tomographic image and the filter map, an image showing a tissue region that is deemed abnormal is generated as the diagnosis map. As described above, the filter map functions as a filter to extract regions that can be a lesion from the tomographic image.

The lesion detector 122 may perform a normalization process on the diagnosis map. The lesion detector 122 performs linear or non-linear conversion on the diagnosis map from the maximum value and minimum value thereof to a range of appropriate values.

The lesion detector 122 may also divide the value of each pixel of the diagnosis map by the feature amount of each pixel of the filter map. This way, the diagnosis map constituted of relative values to the filter map can be obtained.

A diagnosis map 500 of FIG. 5 is generated from the tomographic image 200 of FIG. 2 and the combined filter map 420-3 of FIG. 4C.

Next, the lesion detector 122 detects possible lesions based on the diagnosis map (Step S103). There are various methods to detect possible lesions.

For example, the lesion detector 122 generates an image 510 by binarizing the diagnosis map 500 using a threshold value. The lesion detector 122 detects white sections in the image 510 as possible lesions. The threshold value may be set by the user, or a ratio of the value of the diagnosis map to the maximum value, the average value, or the like may be used.

Next, the lesion detector 122 performs an erroneous detection suppressing process to suppress an erroneous detection of a lesion (Step S104). The lesion detector 122 outputs information for the detected lesions, and ends the process.

For example, the lesion detector 122 narrows down the detected possible lesions based on indexes such as the area and aspect ratio of the region corresponding to each possible lesion, the average value of the diagnosis map, the likelihood calculated by inputting the diagnosis map to a discriminator generated by machine learning, and the like. The lesion detector 122 may narrow down possible lesions by combining a plurality of indexes.

If the possible lesions in the diagnosis map match lesions at a high percentage, the erroneous detection suppressing process does not need to be performed.

In FIG. 5, the lesion detector 122 outputs the image 520 including one lesion out of the three possible lesions detected from the image 510 as the information for the detected lesion.

Next, a method to display the lesion detection result will be explained.

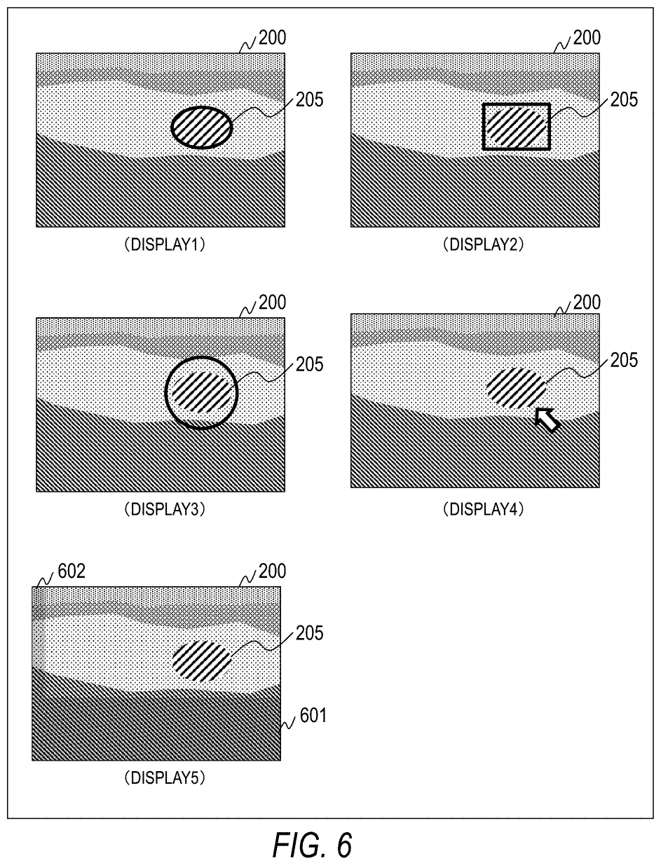

FIG. 6 is a diagram illustrating examples of the lesion detection result presented by the display unit 123 of Embodiment 1.

The display unit 123 generates display data to display images as shown in (Display 1), (Display 2), (Display 3), (Display 4), and (Display 5) as the lesion detection result based on the tomographic image and detection information.

(Display 1) presents a lesion 205 in the tomographic image 200 using the contour form. (Display 2) presents a lesion 205 in the tomographic image 200 using a rectangle enclosing the lesion 205. (Display 3) presents a lesion 205 in the tomographic image 200 using a circle or oval enclosing the lesion 205. (Display 4) presents a lesion 205 in the tomographic image 200 using an arrow pointing at the lesion 205. (Display 5) presents a region 601 and a shadow region 602 of the tomographic image 200 that were excluded from the detection targets.

The display unit 123 may also generate data for causing the output device 109 to output sound and vibration as an alert for the lesion being detected.

As described above, the ultrasound diagnostic apparatus 100 of Embodiment 1 can automatically detect a lesion from a tomographic image. The burden on the medical staff in conducting ultrasonic screening can be reduced. Also, because this makes it possible to prevent oversight of a legion by the medical staff or false detection of a legion, the detection rate of a lesion in ultrasound screening is improved.

### Embodiment 2

In Embodiment 2, the ultrasound diagnostic apparatus 100 analyzes the detected lesion, and presents detailed information of the lesion. Embodiment 2 will be explained below mainly focusing on the differences from Embodiment 1.

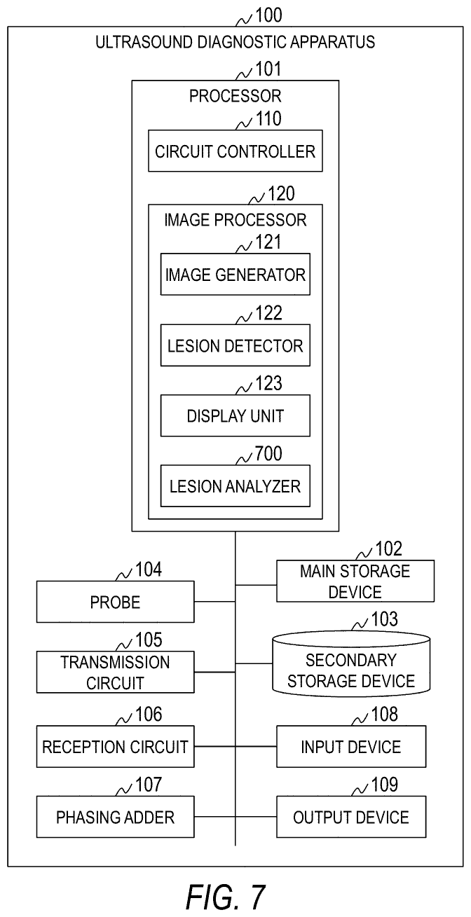

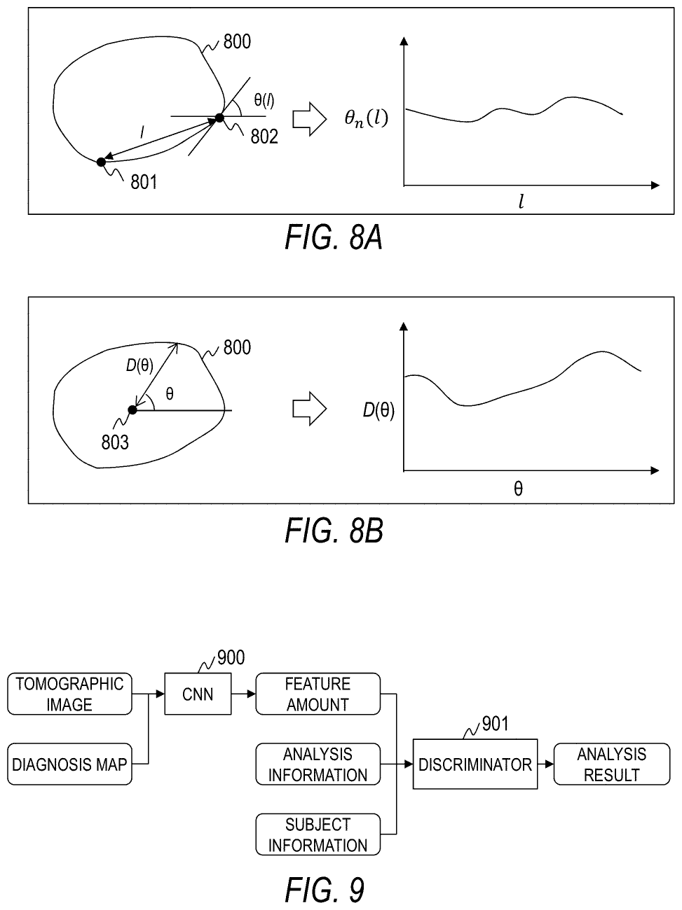

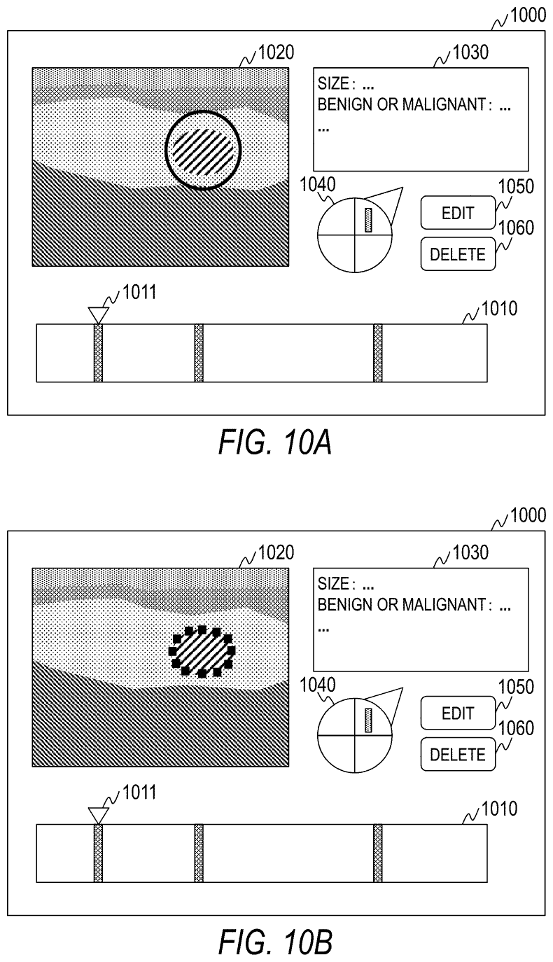

FIG. 7 is a diagram illustrating a configuration example of an ultrasound diagnostic apparatus 100 of Embodiment 2. FIGS. 8A and 8B are diagrams illustrating how the ultrasound diagnostic apparatus 100 of Embodiment 2 analyzes the shape of a lesion. FIG. 9 is a diagram illustrating how the ultrasound diagnostic apparatus 100 of Embodiment 2 determines whether a lesion is benign or malignant, as well as the category thereof.

The hardware configuration and software configuration of the ultrasound diagnostic apparatus 100 of Embodiment 2 are the same as those of the ultrasound diagnostic apparatus 100 of Embodiment 1. However, Embodiment 2 differs from Embodiment 1 in the internal configuration of the image processor 120. Specifically, the image processor 120 of Embodiment 2 further includes a lesion analyzer 700.

The lesion analyzer 700 performs an analysis process on the lesion detected by the lesion detector 122, and outputs the result of the analysis process as analysis information. The lesion analyzer 700 stores, in the secondary storage device 103, the analysis information corresponding to the tomographic image. Below, specific examples of the analysis process will be explained.

(Process B1) The lesion analyzer 700 calculates the border of the lesion based on the area detected as the lesion. The border can be calculated by the threshold process, a process based on the Watershed method, the region dividing method using a discriminator generated by machine learning, or the like.

The lesion analyzer 700 may receive information regarding the border of the lesion specified by the user. In this case, (Process B1) can be omitted.

(Process B2) The lesion analyzer 700 calculates the width and height of the lesion based on the border of the lesion. Further, the lesion analyzer 700 calculates an angle indicating the maximum length using entropy, and calculates the maximum length of the lesion from the angle. The lesion analyzer 700 further analyzes the shape of the lesion border. The border of the lesion can be analyzed based on the complexity, the Fourier descriptor, and the like. The complexity is given by Formula 1.

$$\begin{matrix}Formula 1 & \\ complexity = \frac{{( Perimeter )}^{2}}{Area} & ( 1 )\end{matrix}$$

As illustrated in FIG. 8A, the Fourier descriptor is given as the angle of the tangent at the point of interest 802 at a distance l from the start point 801 when the perimeter of the lesion boundary 800 is L. θ(l) is called a declination function, and generally a coefficient obtained by Fourier series expansion of the normalized declination function shown in Formula 2 is used as the feature amount.

$$\begin{matrix}Formula 2 & \\ {θ}_{n} ( l ) = θ ( l ) - \frac{2 π {r}^{2} l}{L} & ( 2 )\end{matrix}$$

As illustrated in FIG. 8B, a distance D from the center of gravity 803 of the region surrounded by the lesion boundary 800 to the boundary in the direction of the angle θ can also be used as the feature amount.

(Process B3) The lesion analyzer 700 determines whether a legion is benign or malignant, and the category thereof using lesion analysis results such as lesion size, lesion aspect ratio, and border shape, the lesion images, and the like. Examples of the analysis method includes a method using an estimation model generated based on a machine learning algorithm such as logistic regression, support vector machine, random forest, and neural network. The estimation model may be generated by combining a plurality of machine learning algorithms. In a case where a machine learning algorithm with teacher data is used, data composed of tomographic images, lesion detection results, and lesion analysis results may be used as learning data.

Below, the analysis algorithm to determine whether the legion is benign or malignant and the category thereof using a neural network will be described.

The lesion analyzer 700 includes a convolutional neural network (CNN) 900 and a discriminator 901. The discriminator 901 is generated based on a machine learning algorithm such as logistic regression, support vector machine, random forest, and neural network. The discriminator 901 may be generated by combining a plurality of machine learning algorithms.

The lesion analyzer 700 inputs a tomographic image and a diagnosis map to the CNN 900 and calculates a feature amount. Next, the lesion analyzer 700 inputs the feature amount, analysis information, and subject information to the discriminator 901. The discriminator 901 outputs the discrimination result regarding the lesion being benign or malignant, and the category of the lesion.

According to Embodiment 2, the user can view the detection result of a lesion as well as the analysis result of the lesion. This makes it possible to achieve high-quality diagnosis.

### Embodiment 3

In Embodiment 3, the ultrasound diagnostic apparatus 100 presents the lesion detection results and the like in a time-series manner after a series of examinations are completed. Embodiment 3 will be explained below mainly focusing on the differences from Embodiment 2.

The configuration of the ultrasound diagnostic apparatus 100 of Embodiment 3 is the same as that of the ultrasound diagnostic apparatus 100 of Embodiment 2, and therefore, the description thereof is omitted.

In Embodiment 2, the ultrasound diagnostic apparatus 100 outputs the lesion detection result and the lesion analysis result every time after a tomographic image is input. In Embodiment 3, the ultrasound diagnostic apparatus 100 accumulates the lesion detection result and the lesion analysis result in the main storage device 102, and presents the lesion detection results and the lesion analysis results in a time-series manner after a series of examinations are completed.

The detection process of Embodiment 3 is the same as the detection process of Embodiment 1, and the analysis process of Embodiment 3 is the same as the analysis process of Embodiment 2.

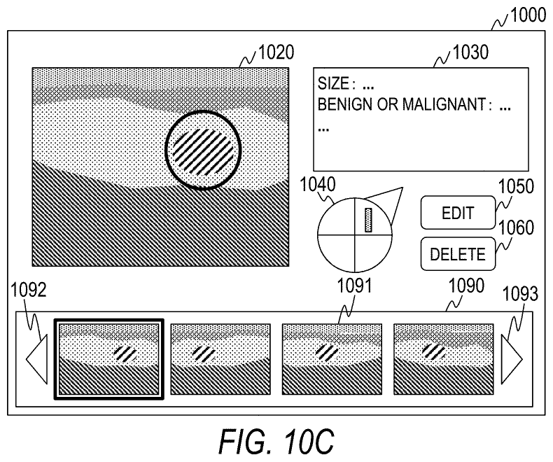

FIGS. 10A, 10B, and 10C are diagrams illustrating an example of the screen displayed by the display unit 123 of Embodiment 3.

The screen 1000 of FIG. 10A includes a time selection field 1010, a detection result display field 1020, an analysis result display field 1030, a positional information display field 1040, an edit button 1050, and a delete button 1060.

The time selection field 1010 is a field to specify the tomographic image to view. In FIG. 10A, a slide bar to specify the time is displayed. In this slide bar, the time corresponding to the tomographic image in which a lesion was detected is highlighted.

The user can select a tomographic image to view by operating a pointer 1011. The display unit 123 obtains a tomographic image of the time corresponding to the pointer 1011, and also obtains detection information and analysis information corresponding to the obtained tomographic image.

The detection result display field 1020 is a field to display the detection information corresponding to the tomographic image of the time selected by the time selection field 1010.

The analysis result display field 1030 is a field to display the analysis information corresponding to the tomographic image of the time selected by the time selection field 1010.

The positional information display field 1040 is a field to display the positional information in the subject at which the tomographic image of the time selected by the time selection field 1010 was obtained. The positional information display field 1040 displays an image or the like based on the spatial position information included in the detection information. For example, in the positional information display field 1040, an image indicating the position of the probe 104 in a breast or the like is displayed.

The edit button 1050 is an operation button to edit at least one of the detection information and analysis information. If the user operates the edit button 1050, the display unit 123 enters the edit mode, and accepts an input into the detection result display field 1020.

For example, in a case where the profile of the lesion is to be corrected, the user operates the detection result display field 1020 as illustrated in FIG. 10B. Specifically, the user sets control points to specify the profile of the lesion. The control points may be set at equal angle, or may be set at change points of the profile. The display unit 123 updates the detection information based on an input of the user. In this case, the display unit 123 may input the detection information back into the lesion analyzer 700 to analyze a lesion again.

The delete button 1060 is an operation button to delete detection information and analysis information. In a case where the user operates the delete button 1060, the display unit 123 deletes detection information and analysis information. In this case, the display unit 123 may input a specific tomographic image 200 into the lesion detector 122 to perform lesion detection again.

The screen 1000 illustrated in FIG. 10C includes a thumbnail display field 1090 instead of the time selection field 1010.

In the thumbnail display field 1090, the thumbnail 1091 and the “next” buttons 1092, 1093 are displayed. If all of the thumbnails 1091 can be displayed within the thumbnail display field 1090, the “next” buttons 1092, 1093 do not need to be displayed.

The user can select a tomographic image to view by selecting the thumbnail 1091. The display unit 123 obtains a tomographic image corresponding to the thumbnail 1091, and also obtains detection information and analysis information corresponding to the obtained tomographic image. Also, as illustrated in FIG. 10C, the display unit 123 may highlight the selected thumbnail 1091.

The layout of the screen 1000 described with FIGS. 10A, 10B, and 10C is merely an example, and the position, size, display method, and the like of the display fields can be appropriately adjusted.

According to Embodiment 3, the user can confirm the lesion detection result and lesion analysis result in a series of examinations, and can edit the results if needed.

The present invention is not limited to the above embodiment and includes various modification examples. In addition, for example, the configurations of the above embodiment are described in detail so as to describe the present invention comprehensibly. The present invention is not necessarily limited to the embodiment that is provided with all of the configurations described. In addition, a part of each configuration of the embodiment may be removed, substituted, or added to other configurations.

A part or the entirety of each of the above configurations, functions, processing units, processing means, and the like may be realized by hardware, such as by designing integrated circuits therefore. In addition, the present invention can be realized by program codes of software that realizes the functions of the embodiment. In this case, a storage medium on which the program codes are recorded is provided to a computer, and a CPU that the computer is provided with reads the program codes stored on the storage medium. In this case, the program codes read from the storage medium realize the functions of the above embodiment, and the program codes and the storage medium storing the program codes constitute the present invention. Examples of such a storage medium used for supplying program codes include a flexible disk, a CD-ROM, a DVD-ROM, a hard disk, a solid state drive (SSD), an optical disc, a magneto-optical disc, a CD-R, a magnetic tape, a non-volatile memory card, and a ROM.

The program codes that realize the functions written in the present embodiment can be implemented by a wide range of programming and scripting languages such as assembler, C/ C++, Perl, shell scripts, PHP, Python and Java.

It may also be possible that the program codes of the software that realizes the functions of the embodiment are stored on storing means such as a hard disk or a memory of the computer or on a storage medium such as a CD-RW or a CD-R by distributing the program codes through a network and that the CPU that the computer is provided with reads and executes the program codes stored on the storing means or on the storage medium.

In the above embodiment, only control lines and information lines that are considered as necessary for description are illustrated, and all the control lines and information lines of a product are not necessarily illustrated. All of the configurations of the embodiment may be connected to each other.

What is claimed is:

1. A diagnostic apparatus diagnosing by using a tomographic image of a subject, comprising:
an image generator configured to generate the tomographic image based on data obtained from the subject; and
a detector configured to perform a process to detect a lesion from the tomographic image,
the detector being configured to:
generate, using the tomographic image, a filter map for extracting a tissue lesion that is potentially abnormal from the tomographic image; and
detect a lesion included in the tomographic image, using the tomographic image and the filter map, and output detection information including a detection result of the lesion.

2. The diagnostic apparatus according to claim 1, wherein the detector is configured to:
set a plurality of analysis layers in an appropriate direction in the tomographic image;
calculate an average value of feature amounts of a plurality of pixels included in the respective plurality of analysis layers; and
generate, as the filter map, an image constituted of a plurality of pixel group layers, each of a plurality of pixels included in one of the plurality of pixel group layers is set an average value of the feature amounts of the plurality of pixels included in one of the plurality of analysis layers.

3. The diagnostic apparatus according to claim 1, wherein the detector is configured to:
generate a diagnosis map by calculating a difference between a feature amount of the tomographic image and a feature amount of the filter map; and
detect a lesion included in the tomographic image based on the diagnosis map.

4. The diagnostic apparatus according to claim 1, further comprising an analyzer configured to analyze the tomographic image in which a lesion was detected,
wherein the analyzer is configured to:
analyze the tomographic image in which a lesion was detected based on an estimation model generated by a learning process using learning data constituted of the tomographic image, the detection result, and an analysis result of the lesion; and
output analysis information including the analysis result of the lesion.

5. The diagnostic apparatus according to claim 4, further comprising a display unit configured to generate display data for presenting the detection information and the analysis information to a user,
wherein the display unit is configured to update at least one of the detection information and the analysis information based on an operation in a case of receiving the operation through an operation screen displayed based on the display data.

6. The diagnostic apparatus according to claim 1, wherein the image generator generates the tomographic image based on data obtained by measuring reflection of ultrasound radiated to the subject.

7. A diagnostic method performed by a diagnostic apparatus diagnosing by using a tomographic image of a subject,
the diagnostic apparatus including: an image generator configured to generate the tomographic image based on data obtained from the subject; and a detector configured to perform a process to detect a lesion from the tomographic image,
the diagnostic method including:
a first step of generating, by the detector generates, by using the tomographic image, a filter map for extracting a tissue lesion that is potentially abnormal from the tomographic image; and
a second step of detecting, by the detector, a lesion included in the tomographic image by using the tomographic image and the filter map, and outputting detection information including a detection result of the lesion.

8. The diagnostic method according to claim 7, wherein the first step includes:
a step of setting, by the detector, a plurality of analysis layers in an appropriate direction in the tomographic image;
a step of calculating, by the detector, an average value of feature amounts of a plurality of pixels included in the respective plurality of analysis layers; and
a step of generating, by the detector, as the filter map, an image constituted of a plurality of pixel group layers, each of a plurality of pixels included in one of the plurality of pixel group layers is set an average value of the feature amounts of the plurality of pixels included in one of the plurality of analysis layers.

9. The diagnostic method according to claim 7, wherein the second step includes:
a step of generating, by the detector, a diagnosis map by calculating a difference between a feature amount of the tomographic image and a feature amount of the filter map; and
a step of detecting, by the detector, a lesion included in the tomographic image based on the diagnosis map.

10. The diagnostic method according to claim 7, wherein the diagnostic apparatus further includes an analyzer configured to analyze the tomographic image in which a lesion was detected, and
wherein the diagnostic method further includes:
a step of analyzing, by the analyzer, the tomographic image in which a lesion is detected based on an estimation model generated by a learning process using learning data constituted of the tomographic image, the detection result, and an analysis result of the lesion; and
a step of outputting, by the analyzer, analysis information including the analysis result of the lesion.

11. The diagnostic method according to claim 10, wherein the diagnostic apparatus further comprises a display unit configured to generate display data for presenting the detection information and the analysis information to a user, and
wherein the diagnostic method further includes a step of updating, by the display unit, at least one of the detection information and the analysis information based on an operation, in a case of receiving the operation through an operation screen displayed based on the display data.

12. The diagnostic method according to claim 7, further including a step of generating, by the image generator, the tomographic image based on data obtained by measuring reflection of ultrasound radiated to the subject.
